(19) United States
(12) Patent Application Publication
(10) Pub. No.: US 2023/0390484 A1
(43) Pub. Date: Dec. 7, 2023

(54) IMPLANTABLE INSULIN PUMP

(71) Applicant: Thunar Medical, Inc., Mount Pleasant, SC (US)

(72) Inventor: Thor Johnson, Mount Pleasant, SC (US)

(21) Appl. No.: 17/831,115

(22) Filed: Jun. 2, 2022

(51) Int. Cl. A61M 5/142; A61B 5/145; A61B 5/1473

(52) U.S. Cl. CPC A61M 5/14212; A61B 5/14532; A61B 5/1473; A61M 5/14276; A61M 2005/1726

(57) ABSTRACT

An implantable insulin pump system and a method for regulating glucose are provided. The system includes a subcutaneous implantable infusion pump having a reservoir of medication, a catheter with an anchor balloon that inflates to engage the portal vein of a patient. The system also includes a glucose measurement device that performs a measurement correlated with blood glucose levels of the patient, and a control unit operatively coupled to the infusion pump and glucose measurement device. The control unit determines the dosage of the medication delivered to the patient. In the method, the catheter tip is implanted in the portal vein and the infusion pump is implanted subcutaneously. The glucose measurement device performs a measurement on a fluid of the patient, and an insulin dosage is calculated by the control unit based on the fluid measurement. The insulin dosage is then delivered into the portal vein.

### TECHNICAL FIELD

The present disclosure is generally related to implantable pumps and methods of regulating glucose.

### BACKGROUND

Diabetes is a group of conditions that results in the body exhibiting abnormally high blood glucose (blood sugar) levels. Diabetes is a result of the pancreas not producing adequate (or any) insulin or the non-pancreatic cells being inadequately responsive to insulin secretion. In patients with type 1 diabetes loss of beta cells leads to inadequate production of insulin. In contrast, in patients with type 2 diabetes, liver and peripheral cells develop decreased response to insulin (insulin resistance) which the pancreas is unable to overcome by increased secretion. This often eventually leads to insulin insufficiency over time.

While there are medications that treat insulin resistance, insulin remains the primary treatment for type 1 diabetes and a very important treatment in type 2 diabetes. Currently, there are several categories of insulin therapies (e.g., short-acting insulin therapy, long-acting insulin therapy) and multiple routes of administration (e.g., injection, pump, inhalation). Daily injections can be painful and cumbersome for patients, as the patients must constantly have medications, injection needles, as well as devices for measuring glucose with them at all times. Insulin pumps are a more recent advance in therapy that are more physiologic in that they deliver constant insulin with the patient increasing the rate of delivery during mealtimes to respond to changes in blood glucose. These still require measuring blood sugar frequently, however, as consistent levels of insulin result in better blood sugar control as demonstrated by A1C levels over time. The disadvantage of pumps is that they are external devices which must be frequently shifted to different locations and can limit patient's normal activities due to the need to protect the pumps from moisture and physical damage, and the patient's requirement to keep pumps on at all times. These pumps administer doses of insulin subcutaneously via a tube and needle or directly through a needle in an adhered pump.

For patients on long-acting insulin, patients must keep track of a specific schedule for insulin in addition to responding to physiologic variations in glucose demonstrated by frequent blood sugar measurements. For patients with a pump, the pump is maintained at a basal level which is then adjusted based on diet and glucose measurement. The necessity for frequent patient measurement and response to blood glucose often leads to missed doses or inappropriately high insulin doses resulting in poor glucose control. This can in some cases lead to life-threatening emergencies. Accordingly, there is a need for improved insulin therapies and insulin administration systems for patients with diabetes. The present application addresses the most important limitations of currently available devices as well as challenges related to insulin therapy and administration of medications more generally.

### SUMMARY

In a first aspect, an implantable insulin pump system is provided. The system includes a subcutaneous implantable infusion pump comprising a reservoir of medication having an output tube, responsive to instructions to deliver a dose of a determined volume of the medication at a defined time. The system also includes a portal vein access catheter comprising: a catheter body having a proximal end, a distal end, a main lumen, wherein the main lumen extends from the proximal end to the distal end, and a balloon-fill lumen, wherein the balloon-fill lumen extends from a balloon-fill hub to an anchor balloon, and the anchor balloon is insertable into a portal vein and inflatable to engage said portal vein and prevent dislodgement. The portal vein access catheter also includes: a subcutaneous connection hub at the proximal end comprising an input port connected to the main lumen and the balloon-fill hub; and a connector that interfaces the main lumen to the reservoir output tube and the balloon-fill lumen to the balloon-fill hub. The system also includes a glucose measurement device configured to perform at least one measurement that is correlated with a patient's blood glucose levels; and a control unit operatively coupled to the infusion pump and the glucose measurement device. The control unit is configured to determine the dosage of the medication to be delivered to the patient and determine a defined time of delivery of the dose via the pump into the portal vein of the patient.

In another aspect, the control unit is implantable. In another aspect, the control unit is located outside of the body of the patient. In another aspect, the medication is insulin or an insulin mimetic. In another aspect, wherein the reservoir is refillable.

In another aspect, at least one of the reservoir, infusion pump, and glucose measurement device is operatively coupled to the control unit via a wire connection.

In another aspect, at least one of the reservoir, infusion pump, and glucose measurement device is operatively coupled to the control unit via a wireless connection.

In another aspect, the glucose measurement device is associated with the catheter and comprises a transducer, and wherein glucose measurement device is configured to perform at least one measurement to determine a patient's blood glucose levels in the portal vein. In a further aspect, the transducer comprises two leads secured proximate to the tip of the catheter, wherein the leads are configured to measure impedance in the blood flow of the patient, which is correlated with the patient's blood glucose levels. In a further aspect, the leads of the glucose measurement device run the length of the catheter and attach to the control unit. In another aspect, the glucose measurement device is a micro circuit and the transducer is integrated into the catheter. In a further aspect, the transducer is integrated into the micro circuit and the micro circuit is located at the tip of the catheter.

In another aspect, the glucose measurement device is an impedance sensor that is configured to measure an impedance in the blood flow which is correlated to a patient's blood glucose levels, and wherein the glucose measurement device or the control unit is configured to calculate a blood glucose level of the patient based on the impedance measurement.

In another aspect, the glucose measurement device is a fingerstick glucose device and an accompanying blood glucose meter. In a further aspect, the fingerstick glucose device comprises a lancet and a test strip, and wherein the blood glucose meter is operatively coupled to the control unit and configured to send glucose measurements to the control unit.

In another aspect, the glucose measurement device is a continuous glucose monitoring sensor. In a further aspect, at least a portion of the continuous glucose monitoring sensor is positioned subcutaneously and said portion is in fluid connection with the interstitial fluid of the patient. In another aspect, the continuous glucose monitoring sensor is operatively coupled to the control unit and configured to send at least one measurement to the control unit that is correlated with the patient's blood glucose levels.

In another aspect, the system further comprises an adaptor configured to connect the catheter to the infusion pump and keep the anchor balloon inflated.

In another aspect, the subcutaneous connection hub further comprises an infusion lumen port connected to said main lumen.

In a second aspect, a method for regulating glucose in a subject with the implantable insulin pump system is provided. In the method, at least a tip portion of a portal vein access catheter is implanted within a portal vein of a patient. An infusion pump is implanted subcutaneously, and the infusion pump is coupled to the catheter. A measurement on a fluid of the patient is then conducted with the glucose measurement device, wherein the fluid is interstitial fluid or blood, and wherein the measurement is correlated with a blood glucose level of the patient. A dosage of insulin to deliver to the patient is then calculated by the control unit based on the measurement on the fluid of the patient. The calculated dosage of insulin is then delivered, by the infusion pump, to the tip of the portal vein access catheter, wherein the tip of the portal vein catheter is in fluid connection with the portal vein. The insulin is then expelled by the portal vein access catheter from the tip of the catheter into the portal vein.

In another aspect of the method, a portion of the delivered insulin mixes with a portion of the patient's blood in the tip of the catheter prior to being expelled into the portal vein.

### BRIEF DESCRIPTION OF THE DRAWINGS

FIG. 1 illustrates an exemplary direct portal venous access catheter of the implantable insulin pump system in accordance with one or more embodiments.

FIG. 2 illustrates an exemplary transjugular venous access catheter of the implantable insulin pump system in accordance with one or more embodiments.

FIG. 3A illustrates an exemplary infusion pump and an exemplary control unit of the of the implantable insulin pump system in accordance with one or more embodiments.

FIG. 3B illustrates an exemplary infusion pump and portal venous catheter of the implantable insulin pump system in accordance with one or more embodiments.

FIG. 3C illustrates an exemplary infusion pump and portal venous catheter of the implantable insulin pump system in a portal vein in accordance with one or more embodiments.

FIG. 4 illustrates a flow diagram of an exemplary method for regulating glucose in a subject with the implantable insulin pump system in accordance with one or more embodiments.

### DETAILED DESCRIPTION

In accordance with one or more embodiments, the present application describes an implantable insulin pump system and related methods. In one or more embodiments, the implantable insulin pump system is a closed-loop system that responds directly to glucose levels without patients being required to interact with the device to either measure glucose or administer insulin. As there are presently no commercially available completely implantable insulin pumps, the present system overcomes many of the shortcomings of conventional pumps, including allowing patients to swim or engage in other physical activity with significantly fewer limitations than current devices.

The implantable insulin pump system can include a portal vein access catheter having a catheter body, a balloon-fill lumen, and an anchor balloon. The anchor balloon is configured to be inserted into the portal vein of a patient and is inflatable such that it engages the portal vein and prevent dislodgements.

The system also includes a subcutaneous implantable infusion pump that comprises a reservoir of medication (e.g., insulin, insulin mimetic) and that is responsive to signals to deliver a dose of a determined volume of the medication at a defined time. The system also includes a glucose measurement device (e.g., impedance sensor) that is coupled to the catheter. In one or more embodiments, the glucose measuring device can comprise two leads secured near to or at the tip of the portal vein access catheter, and the leads are configured to measure the blood glucose levels of the patient or an impedance in the blood flow, which is correlated to the patient's blood glucose level. The system further includes a control unit that is operatively coupled to the infusion pump, the reservoir, and the glucose measurement device. The control unit is configured to determine the dosage of the medication to be delivered to the patient based on the determined blood glucose level and determine a defined time of delivery of the medication. The control unit can then be configured to send a signal to the infusion pump to deliver the determined dosage of (an effective amount of) medication to the catheter and ultimately to the patient's bloodstream via the portal vein.

The present implantable insulin pump system imitates the functions of a healthy pancreas with respect to the production and release of insulin in response to blood glucose levels. Additionally, because the system regularly measures glucose and delivers specifically calculated insulin doses into the portal vein, the amount of insulin required is substantially reduced compared to conventional subcutaneous injections, nasal inhalation systems, or external insulin pump systems. Specifically, physiologic secretion of insulin in humans is typically between 2-10 picomoles/kg/min from an intact pancreas. Conventional therapy with subcutaneous insulin in patients with a type 1 diabetes is approximately 0.3 units per kg per day (a unit of insulin is 0.34 grams and 1 gram of insulin is 0.00246 moles). In current clinical practice, the subcutaneous delivery of insulin requires 177,538% more insulin than physiologic insulin secretion by the pancreas into the portal vein. In contrast, due to the implantation of the catheter of the present system into the portal vein, delivery of insulin with the present system requires approximately 4 orders of magnitude less medication than insulin delivery subcutaneously.

Moreover, by being implanted in the portal vein, the present system can provide faster response times for controlling glucose levels with less fluctuations between doses compared to conventional insulin treatments. The present system in the portal vein also generally results in better glucose control and decreased A1C levels relative to intermittent subcutaneous injections.

These and other aspects of the present systems and methods are described in further detail below with reference to the drawing figures.

### Definitions

The term “about” or “approximately” as used herein, in the context of any of any assay measurements refers to +/−5% of a given measurement.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

As discussed above, in one or more embodiments, the present implantable insulin pump system includes a portal vein access catheter (portal venous catheter). In one or more embodiments, the portal vein access catheter is a portal venous access catheter as shown and described in commonly assigned U.S. Pat. No. 11,147,946 issued Oct. 19, 2021, U.S. application Ser. No. 17/317,676, filed May 11, 2021, U.S. application Ser. No. 17/317,717, filed May 11, 2021, and U.S. Application Ser. No. 17/501,148, filed on Oct. 14, 2021, all of which are hereby incorporated by reference in their respective entireties.

In one or more embodiments, the portal venous access catheters contemplated herein are an improvement over conventional catheters in that the presently disclosed transveous approach allows a second lumen within the systemic venous circulation in addition to the portal lumen. In one or more embodiments, the presently disclosed catheters differ from conventional catheters in design in that they comprise a distal anchor balloon providing advantages including, but not limited to: i) occlude the catheterized vessel; and ii) prevent respiratory motion-induced dislodgement (an unavoidable problem when using conventional catheters).

In at least one embodiment, the portal venous catheter comprises the hepatic portal venous catheter. In one embodiment, an abdominal percutaneous insertion site provides access to the hepatic portal venous system. In at least one embodiment, the portal vein catheter is a temporary catheter. In at least one embodiment, the catheter is a long-term catheter. In at least one embodiment, the catheter is a long-term tunneled catheter. In at least one embodiment, an internal jugular insertion site provides access to the hepatic portal venous systems. In at least one embodiment, the internal jugular insertion site further comprises a hepatic vein.

Unlike conventional venous infusion catheters, the portal vein catheter of the present system is configured with an anchoring balloon. In some embodiments, the present portal vein catheter contemplates a catheter design that provides for a customized “sized fit” by tailoring each catheter to a different size. In at least one embodiment, a tissue retention sheath of a portal access tunneling catheter is separate from the catheter body. Consequently, the tissue retention sheath, in conjunction with a connection hub, can be inserted into a tailored catheter wherein a tissue retention cuff remains peripheral to the entry site. In one or more embodiments, portal venous access catheters contemplated herein are constructed out of polymer materials currently available and used in conventional catheters. In other embodiments, the catheters are heparinized.

### A. Direct Portal Venous Access Catheters

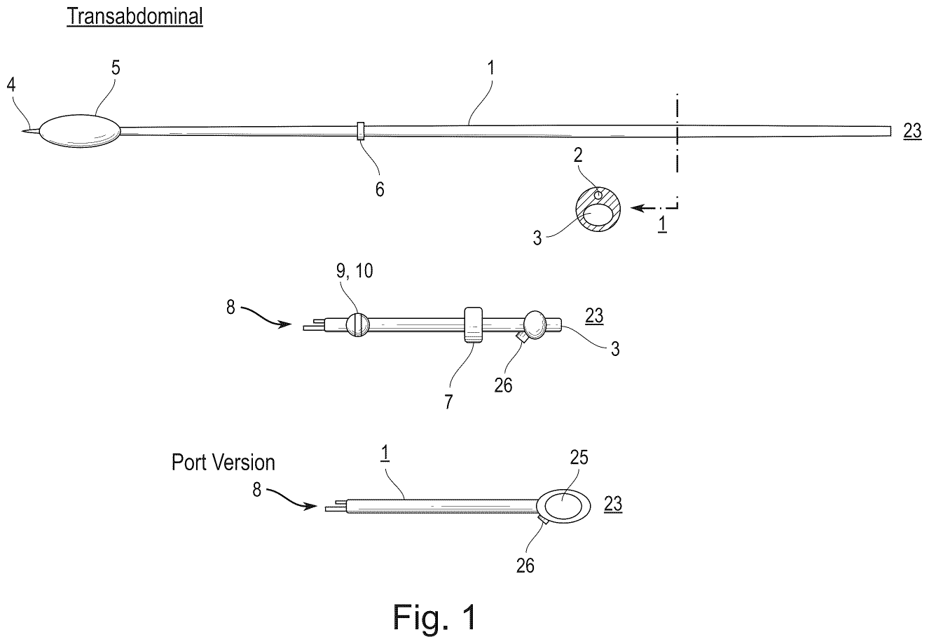

In some embodiments, the present system utilizes a direct portal venous access catheter. In other embodiments, a central venous access catheter may be used. FIG. 1 illustrates an exemplary direct portal venous access catheter in accordance with one or more embodiments. With particular reference to FIG. 1, in accordance with one or more embodiments, the portal vein catheter includes a catheter body (1) that is configured with a balloon-fill lumen (2) and a main lumen (3). Attached to the distal tip (4) of the catheter body (1) is an inflatable anchor balloon (5). A ring slide (6) or hemostatic clamp (7) surrounds the catheter body (1) and may be moved longitudinally to secure to a metal connector (8) protruding from the proximal end of the catheter body (1). A tunnel tissue ingrowth cuff (9) is attached to the proximal end of the catheter body (1) proximate to the metal connector (8). In other embodiments, the tunnel tissue ingrowth cuff (9) is replaced with suture slots (10). In a port version shown in FIG. 1, the proximal end (23) of the catheter body (1) has a subcutaneous port (25) connected to the main lumen (3) and a balloon-fill port (26) connected to the balloon-fill lumen (2). The balloon-fill port (26) is oriented perpendicular to the subcutaneous port (25). In this port version, the entire catheter body (1) is implantable with at least the anchor balloon and the distal tip located in the portal vein.

### B. Transjugular Portal Venous Access Catheters

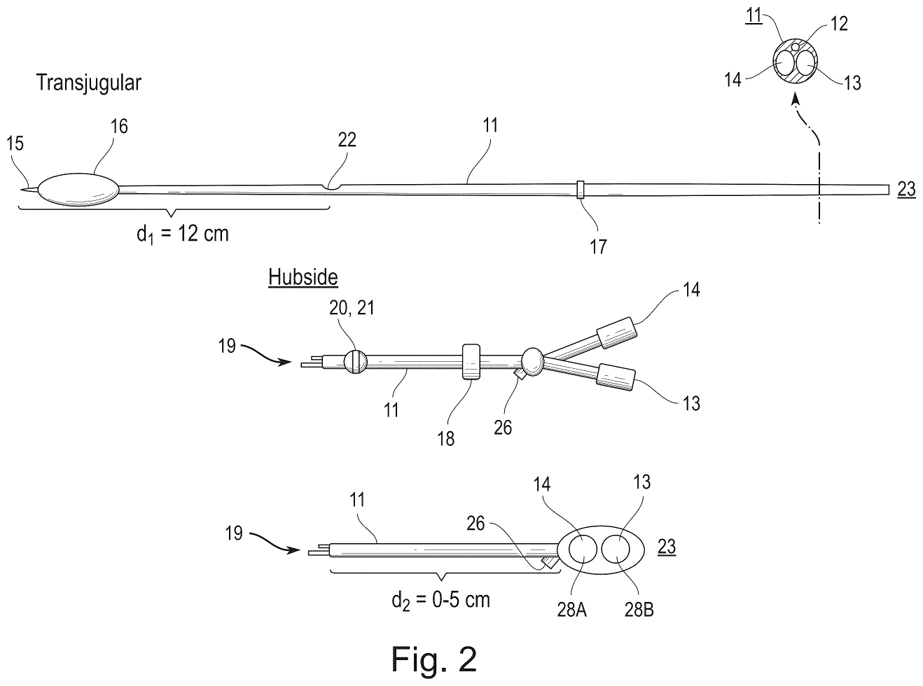

In some embodiments, the present system utilizes a transjugular portal venous access catheter. FIG. 2 illustrates an exemplary transjugular portal venous access catheter in accordance with one or more embodiments. With particular reference to FIG. 2, in accordance with at least one embodiment, the transjugular portal venous access catheter includes a catheter body (11) is configured with a balloon-fill lumen (12) a portal venous lumen (13) and a central venous lumen (14). Attached to the distal tip (15) of the catheter body (11) is an inflatable anchor balloon (16). A ring slide (17) or hemostatic clamp (18) surrounds the catheter body (11) and may be moved longitudinally to secure to a metal connector (19) protruding from the proximal end of the catheter body (11). The catheter body (11) bifurcates to separate the portal venous lumen (13) from the central venous lumen (14) and the balloon-fill lumen (12). A tunnel tissue ingrowth cuff (20) is attached to the proximal end of the catheter body (11) proximate to the metal connector (19). In other embodiments, the tunnel tissue ingrowth cuff (20) is replaced with suture slots (21). A transjugular catheter is shown having the central venous lumen (14) terminate at a cutaneous end hole (22) a distance d₁, e.g., approximately 12 centimeters, from said distal end (15) of the catheter body (11). In a port version shown in FIG. 2, the proximal end (23) of the catheter body (11) has a subcutaneous port (28) with a first access port (28A) connected to the central venous lumen (14) and a second access port (28B) connected to the portal venous lumen (13) and a balloon-fill port (26) connected to the balloon-fill lumen (12). The balloon-fill port (26) is oriented perpendicular to the subcutaneous port (25). The metal connector (19) may be spaced a distance d₂ between 0 and 5 cm from the subcutaneous port (28). In this port version, the entire catheter body (1) is implantable with at least the anchor balloon and the distal tip located in the portal vein.

Thus, in one or more embodiments, the portal vein access catheter can comprise a catheter body having a proximal end, a distal end, and a main lumen extending from the proximal end to the distal end. The catheter can also include a balloon-fill lumen and an anchor balloon, where the balloon-fill lumen extends from a balloon-fill hub to the anchor balloon. The anchor balloon is insertable into the portal vein of the subject and inflatable to engage said portal vein and prevent dislodgement. The medication of the present implantable insulin pump system is dispensed within the patient via the tip of the portal vein catheter.

In one or more embodiments, the catheter can be inserted in either the left or right portal vein. In at least one embodiment, the main lumen of the catheter can be in the main portion of the portal vein, but in other embodiments, it can be inserted in many different places within the portal vein, such as generally either right anterior portal vein, right posterior portal vein, the segment 3 portal vein, or other tributaries of the portal vein. In at least one embodiment, the subcutaneous connection hub (subcutaneous port (25) or (28)) of the catheter can further includes a subcutaneous infusion lumen port connected to the main lumen, so that the patient can be injected with medicine directly into the portal vein through the already installed catheter, without have to go through the infusion pump (or its reservoir).

In addition to the catheter, in one or more embodiments, the present implantable insulin pump system also comprises a subcutaneous implantable infusion pump. Unlike the implantable infusion pump of an embodiment of the present system, conventional infusion pumps are generally external to the body of the subject, which can result in issues for the subject, ranging from minor annoyances to medical emergencies. For example, conventional infusion pumps generally stay connected to the outside of the body of the patient (should not be disconnected for more than about 30 minutes) and cannot be submerged in water. As such, certain types of activities, such as swimming, must be avoided by patients with a conventional infusion pump. Even everyday activities, such as a bath or shower, must be done with caution by removing the infusion pump for a short period or ensuring that the needle and injection site stay dry. Additionally, conventional infusion pumps required needle access to the skin, and the needle must be moved every 1-3 days. This can result in soreness and scarring at the various injection sites, and because of the soreness, can sometimes result in non-compliance by the patient, leading to poor glucose control. Moreover, because patients oftentimes are not medical professionals themselves and do not use the proper precautions with needles, the regular removal and insertions of needles by the patient carries an increased risk of infection. The tubing of conventional infusion pumps also carries an increased risk of infection if not cleaned properly.

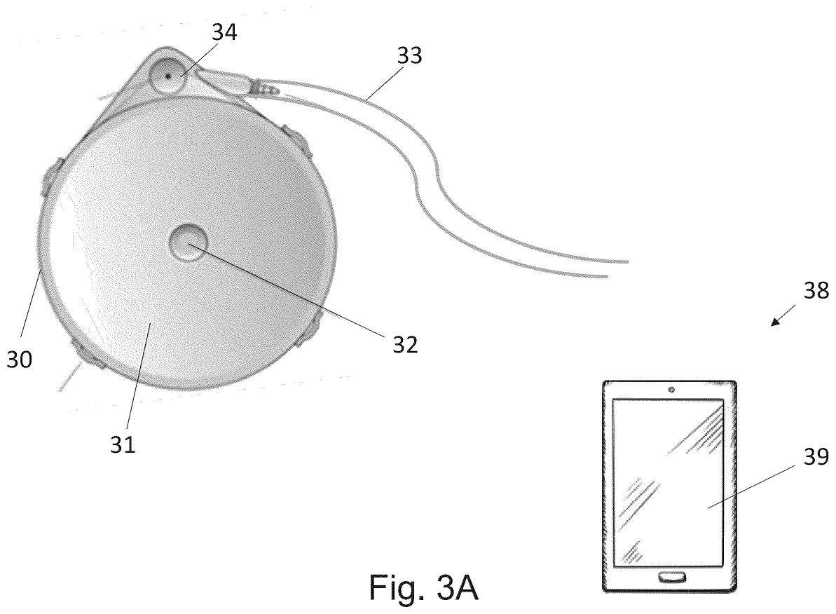

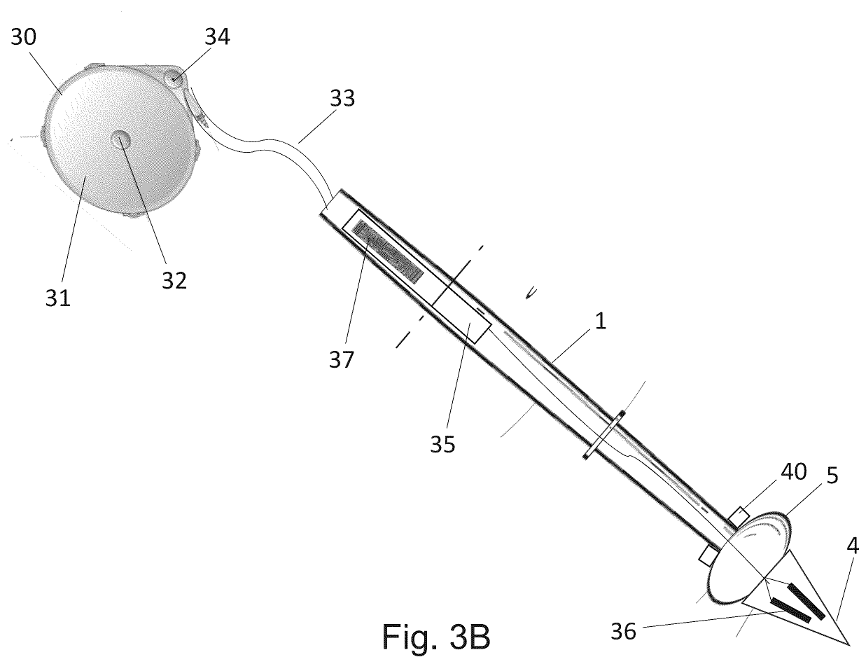

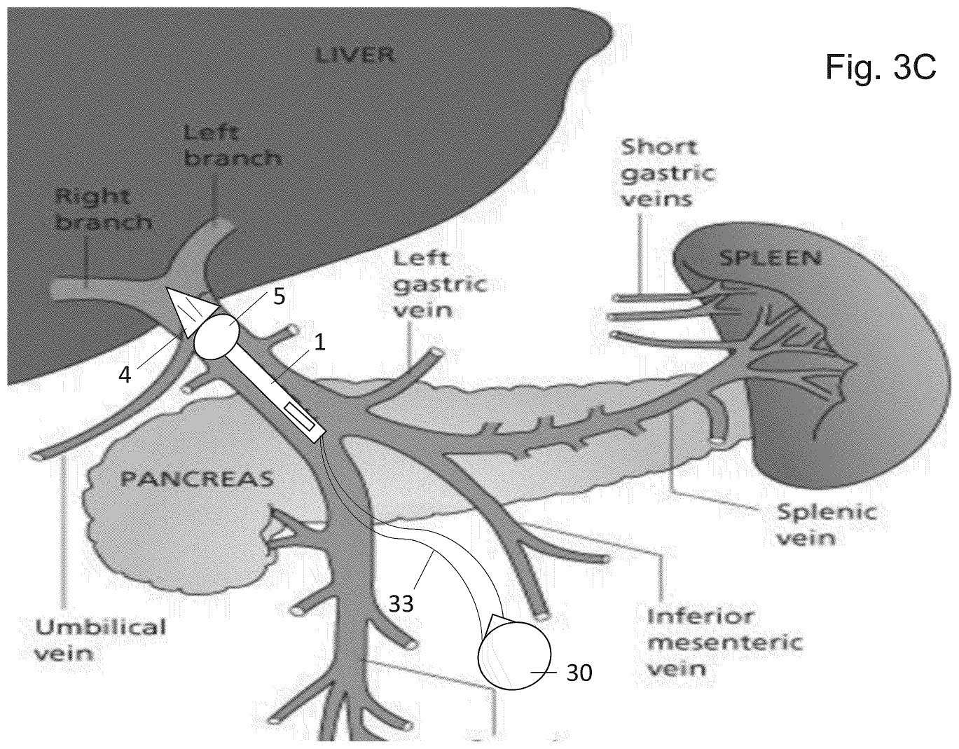

The subcutaneous implantable infusion pump of the present system addresses many of the deficiencies of conventional infusion pumps. FIGS. 3A-3C provide diagrams of an exemplary implantable insulin pump systems, including the implantable infusion pump, in accordance with one or more embodiments. Specifically, in one or more embodiments, the implantable infusion pump system is implantable subcutaneously in the body of the patient and secured against movement. More specifically, in one or more embodiments, the infusion pump is implantable in a left lower quadrant or right lower quadrant or flank of the patient's body. By being implantable within the body of the patient, the present infusion pump avoids the inconvenience and risks of repeated disconnection and re-connection of the infusion pump during prolonged exposure to water (e.g., shower, bath, swimming). The implantation of the present infusion pump also negates the need for regular injections by the patient, thereby avoiding injection site soreness and limiting infection risk. By avoiding the regular need for needles, the implantable infusion pump of the present system can also result in significant cost savings to the patient and a reduction in biomedical waste that is typically associated with daily injections.

With reference to FIGS. 3A-3C, the subcutaneous implantable infusion pump 30 of the present system includes a reservoir 31 of medication. In one or more embodiments, the medication is a type of insulin or an insulin mimetic (insulin analog), both of which can sometimes include a pharmaceutically acceptable carrier or at least one pharmaceutically acceptable additive. In one or more embodiments, the reservoir 31 is refillable via a reservoir fill port 32 on an outer surface of the infusion pump 30. In one or more embodiments, the subcutaneous infusion pump 30 can include an access membrane in communication with the fill port 32. To refill the reservoir 31, the access membrane can be punctured through the skin, such that the old medication can be aspirated, and then new medication can be placed in the reservoir 31 via the fill port 32. In addition, there can be a second port spaced away from the fill port 32 that allows direct access to the catheter portion of the device without infusion through the reservoir. The reservoir fill port 32 can be connected to the reservoir 31 via a valve and a conduit, for example. For instance, in one or more embodiments, insulin can enter the pump 30 through the reservoir fill port 32 and pass through the conduit and/or valve to reach the reservoir 31.

In one or more embodiments, the volume of medication that the reservoir 31 can hold is correlated to the stability of the medication and the likely number of doses to be delivered between refills.

In one or more embodiments, the infusion pump 30 is connected to the portal vein access catheter via output tube 33, optionally one or more valves, and an access port 34 which allows the insulin to pass from the reservoir 31 of the infusion pump 30 to the catheter. In one or more embodiments, the infusion pump 30 applies pressure on the reservoir 31 via pressurized gas to release the insulin from the reservoir 31 into the output tube 33 and through the valve and/or access port to reach the catheter. In one or more embodiments, the reservoir 31 is integrated into the pump 30 and is sized to hold an appropriate amount of insulin based on the needs of the patient for a desired period of time in light of the useful life (stability) of the insulin stored under such conditions. In one or more embodiments, the infusion pump 30 is a SYNCHROMED™ II intrathecal pump produced by MEDTRONIC or a modified version thereof. In at least one embodiment, the infusion pump is a FLOWONIX infusion pump or a modified version thereof. In one or more embodiments, the infusion pump includes a battery having a long lifespan. For example, in at least one embodiment, the battery of infusion pump can last approximately 7-10 years (e.g., SYNCHROMED™ II intrathecal pump; FLOWONIX infusion pump).

In at least one embodiment, the pump is positioned outside of the body and is not implantable. In such an embodiment, the pump can be selectively and removably attached to the catheter, and the catheter can be a tunneling catheter and such a system can obtain many of the advantages of the system having an implantable pump.

With continued reference to FIGS. 3A-3C, the system of the present application further includes a glucose measurement device 35 that is coupled to the catheter. In one or more embodiments, the glucose measurement device 35 is configured to perform at least one measurement to determine a patient's blood glucose levels, such as a direct measurement of blood glucose or a measurement of impedance in the blood flow which is correlated to a patient's blood glucose levels. In one or more embodiments, the glucose measurement device is a transducer and is configured to perform at least one measurement to determine a patient's blood glucose levels. As used herein, a “transducer” is a device that makes a measurement of a blood parameter that can be correlated, directly or indirectly, to blood glucose level.

In one or more embodiments, the transducer comprises two electrical leads secured proximate to the tip of the catheter, and the electrical leads are configured to measure impedance in the blood flow of the patient, which is correlated with the patient's blood glucose levels as is known in the art. Specifically, in one or more embodiments, the glucose measurement device 35 (or transducers) comprises an impedance sensor. In one or more embodiments, the glucose measuring device 35 comprising two leads 36 that are secured at or near the tip 4 of the catheter and the portal vein catheter can be modified to accommodate the leads 36. The leads 36 can run along at least a portion of the length of the catheter. In one or more embodiments, the leads run along the entire length or substantially the entire length of the catheter. The leads run back to sensing circuitry 37 of the glucose measurement device 35. In one or more embodiments, the leads 36 are external to the body of the catheter. In at least one embodiment, the leads 36 are located within the body of the catheter. For example, the leads can be run through the main lumen of the catheter or integrated into the catheter body.

In at least one embodiment, the glucose measurement device 35 is a micro circuit and the transducer is integrated into the catheter. In at least one embodiment, the transducer is integrated into the micro circuit and the micro circuit is located at the tip of the catheter.

In embodiments in which the glucose measurement device 35 is an impedance sensor, the leads 36 are configured to measure an impedance in the blood flow which is correlated to a patient's blood glucose levels. Specifically, the two leads 36 are secured to the tip 4 of the catheter to measure impedance in the portal vein of the patient. The glucose measurement device 35 is also operatively coupled to a control unit 38 of the system via a wired connection or a wireless connection. Upon measurement of the impedance in the blood flow, the measured impedance value can be transmitted by the sensing circuitry 37 of the glucose measurement device 35 to the control unit to calculate the corresponding blood glucose level of the patient. In at least one embodiment, the glucose measurement device 35 itself is configured to calculate the corresponding blood glucose level from the impedance measurement. It should be understood that from time to time it may be useful to calibrate the glucose level as determined based on an impedance measurement in the portal vein based on the patient's glucose level as measured by a conventional blood glucose measurement based on a contemporaneously extracted blood sample, e.g., a fingerstick based measurement. Such a calibration may be factored into the determination of the blood glucose level based on the impedance resistance measurement.

The control unit 38 can then determine the dosage of insulin to be delivered to the subject and the timing of delivery of the dosage of insulin. In one or more embodiments, the glucose measurement device is a GUARDIAN SENSOR 3 produced by MEDTRONIC or a modified version thereof.

As mentioned above, the present system comprises a control unit 38 that is operatively coupled to the infusion pump 30, the glucose measurement device 35, and optionally the reservoir 31 of the infusion pump 30. In one or more embodiments, the control unit 38 can be physically connected to the infusion pump 30 or the glucose measurement device 35 and implanted within the body of the subject. In at least one embodiment, the control unit 38 is external to the body (e.g., a computing device) and wirelessly connected to the infusion pump 30 and other aspects of the system. In one or more embodiments, the signals and measurements transmitted and received by the control unit can be monitored by the patient via a program executed by a computing device (e.g., an app on a smartphone). In one or more embodiments, the control unit 38 is configured to calculate a blood glucose level of the patient based on the measured impedance value and determine the corresponding dosage of the medication (e.g., insulin) to be delivered to the patient. The control unit 38 is then configured to transmit a signal to the infusion pump 30 and/or the reservoir 31 to release the determined dosage of medication (effective amount of medication) from the reservoir 31, and the determined dosage is expelled from the reservoir 31 through output tube 33 to the tip 4 of the catheter. In other words, at least one portion of the infusion pump 30 (e.g., the reservoir, or the pump itself) can be operatively coupled to the control unit 38 of the present system and can transmit to and receive signals from the control unit 38. For example, the pump 30 can be responsive to instructions from the control unit 38 including instructions to deliver a dose of a determined volume of the medication at a defined time or plurality of times. The connection between the control unit 38 and the implantable infusion pump can be a wired connection or a wireless connection.

The control unit 38 can also be operatively connected to a display 39 for the patient. The display 39 could be a display of a smartphone, for example, that is configured to run an app for interacting with the present system via wireless connection (e.g., Bluetooth, NFC, Wi-Fi). The processor of the smartphone, in executing the app, can be configured to display, for example, impedance measurements made by the system, corresponding blood glucose values, or both, dosages of medication dispensed by the reservoir, and the time and date of each measurement and delivery of the medication. The app can also be configured to display or sound an alarm on the smartphone to let the patient know if an error or malfunction has occurred (e.g., pump malfunction), if the patient's blood glucose is above or has dropped below a certain threshold, or if the reservoir is becoming low on medication (insulin), for example. In at least one embodiment, the control unit 38 can be recalibrated for patients with specific complications (e.g., significant risk of hypoglycemia) such that the blood glucose thresholds for alerting the patient are changed to more specifically suit the patient.

After the medication is expelled from the reservoir 31, the medication then flows through output tube 33 into the main lumen of the portal vein catheter and then is released from the tip 4 of the catheter into the bloodstream of the patient. In one or more embodiments, the tip 4 of the catheter is in fluid communication with the portal vein such that the medication in the tip can mix with blood in the portal vein before the dose of medication is forcefully expelled out the catheter tip into the portal vein. In other words, there is natural mixing of the blood and the medication in the tip 4 in the portal vein due to turbulent flow, and the medication can be injected into the portal vein via the tip 4 in a pulsatile fashion. This operates to modulate the dosage delivery to limit the spike of medication (e.g. insulin), and thereby allow for better or smoother absorption of the medication in the liver.

In one or more embodiments, an adaptor 40 can be attached to the catheter to ensure that the lumen supporting the anchor balloon keeps the balloon properly inflated for the entire time that the catheter is in place in the portal vein (e.g., for the battery lifespan of the device, 7-10 years). Normally the catheter remains in place and the anchor balloon generally remains inflated because it has a one-way fill valve such that once it is filled it is and it stays inflated. In instances in which the balloon begins to undergo deflation, the one-way fill valve can be accessed again to add more air to the balloon. Additionally, the anchor balloon is also unlikely not fall out of place once it is in a position for greater than 6 weeks because fibrin forms along the catheter which keeps it from falling out place. In one or more embodiments, the adaptor 40 allows for the delivery of the medication (e.g., insulin) via the tip of the catheter into the portal vein without the tip “milking” out of position. In other words, as the patient breathes, the patient's liver moves up and down, but the chest wall is fixed. As such, if the catheter is moving back and forth with liver movement, it is possible that the catheter can fall out of the portal vein into the peritoneum because of this movement, which is considered “milking” out of position. The adaptor 40 allows for the medication to be delivered into the portal vein without the tip of the catheter “milking” out of position.

As described above, in one or more embodiments, the present system can conduct glucose measurements via the glucose measurement device 35 (e.g., an impedance sensor) that is operatively attached to the catheter and provided in the portal vein of the patient. However, in one or more embodiments, one or more other techniques can be used for measuring glucose levels of the patient, include techniques in which the blood glucose is measured at a location remote from the portal vein, and remote from the catheter and the control unit. For example, blood glucose measurements can be made via a fingerstick glucose device (e.g., lancets and test strips) and an accompanying blood glucose meter, as is known in the art. In one or more embodiments, the blood glucose meter can be operatively coupled (e.g., wirelessly) to the control unit and configured to send glucose measurements to the control unit. Upon receiving the glucose measurements from the blood glucose meter, the control unit can transmit signals to the pump including instructions for dose delivery of the medication. In an exemplary embodiment, the blood glucose meter can be a Contour Next Link 2.4 Meter as produced by Ascensia Diabetes Care Holdings AG or a modified version thereof.

Further, in at least one embodiment, the glucose measurement device 35 can be remote from the portal vein of the patient and can measure the blood glucose level of the subject via their interstitial fluid. For instance, in at least one embodiment, the glucose measurement device can be a continuous glucose monitoring sensor. In one or more embodiments, the continuous glucose monitoring sensor can be attached to the patient's skin such that a portion of the sensor is partially beneath the skin and said portion is in fluid connection with the interstitial fluid of the patient. In one or more embodiments, the continuous glucose monitoring sensor can be a completely subcutaneous sensor. The sensor can measure the patient's blood glucose levels at regular time intervals and record or store the glucose measurements. The sensor can be operatively coupled (e.g., wirelessly) to the control unit and configured to send glucose measurements to the control unit. Upon receiving the glucose measurements, the control unit can transmit signals to the pump including instructions for dose delivery of the medication.

Alternatively, the sensor can be configured send a measurement or data that can be subsequently correlated to a glucose measurement by the control unit or a remote processor (e.g., mobile device configured to run a corresponding app). For example, the sensor can be configured to make a measurement in the interstitial fluid of the patient, and then send the measurement (data) to control unit (or a remote processor). The control unit (or the remote processor) can then be configured to correlate the measurement or data into a corresponding glucose level of the patient. The control unit (or the remote processor via the control unit) can then be configured to transmit the corresponding glucose measurement to the pump, including instructions for dose delivery of the medication. In yet another alternative embodiment, the measurement or data made by the sensor, which can be correlated to a glucose measurement by the control unit, is not converted to a glucose level by the control unit or the remote processor, but rather is send directly to the infusion pump which, based on the measurement, can set the corresponding dose of medicine (e.g., insulin) without first converting the measurement to a glucose level.

In at least one embodiment, any determined glucose measurements can also be sent to a display (e.g., display of a mobile device operating a corresponding app) for the benefit of the patient.

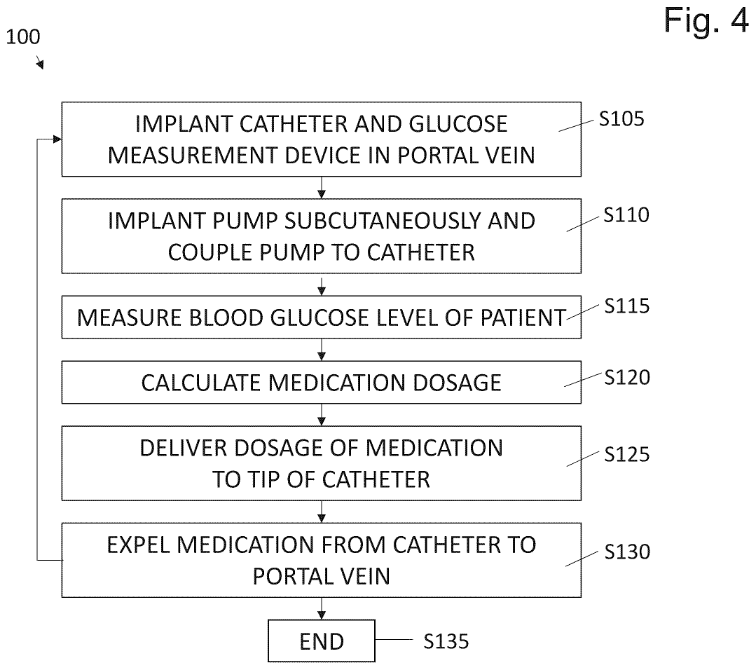

In an exemplary embodiment, the continuous glucose monitoring sensor can be the Freestyle Libre 2 as produced by Abbott Laboratories or a modified version thereof. In another exemplary embodiment, the continued glucose monitoring sensor can be the sensor of the Eversense E3 Continuous Glucose Monitoring system as produced by Senseonics, Inc and Ascensia Diabetes Care Holdings AG, or a modified version thereof. Accordingly, the present system can be used to perform methods for regulating glucose in a subject, such as a patient with diabetes. For instance, a flow diagram of an exemplary method for regulating glucose in a subject with the implantable insulin pump system is shown at FIG. 4 in accordance with one or more embodiments. With reference to FIG. 4, in one or more embodiments, to begin the method 100, at step S105 at least a distal portion of the portal vein access catheter and at least a portion of the glucose measurement device are implanted within a portal vein of a patient. At step S110 the infusion pump is implanted subcutaneously and coupled to the catheter. In one or more embodiments, steps S105 and S110 can be combined or performed at approximately at the same time. The control circuit may be implanted or not, and the signal processing portion of the glucose measurement device also may be implanted or not, as discussed in this disclosure. Upon implantation, at step S115 a blood glucose level of the patient is measured by the glucose measurement device. Alternatively, as mentioned above, in one or more embodiments, the glucose measurement device can perform a measurement on a fluid of the patient (e.g., interstitial fluid or blood), and the measurement can be correlated with the blood glucose level of the patient but is not itself a measurement of blood glucose. At step S120, a dosage of medication (e.g., insulin) is then calculated by the control unit based on the measurement of the glucose measurement device (e.g., blood glucose level). After the dosage of medication (e.g., insulin) is determined, at step S125 the calculated dosage of the medication is delivered by the infusion pump through the output tube 33 and main lumen to the tip of the portal vein access catheter. The tip of the portal vein catheter is in fluid communication with the portal vein. In one embodiment a portion of the delivered insulin mixes with a portion of the patient's blood within the tip of the catheter. Finally, after delivery to the tip of the catheter, at step 130 the insulin is expelled by the portal vein access catheter into the portal vein of the patient. This method can be repeatedly performed by the system multiple times per day, on a set schedule or via initiation by the patient or a medical professional via a signal from a computing device to the control unit, for example. The method ends at step S135.

The present implantable insulin pump system provides many advantages over the current system, including current diabetes treatment systems. For example, the battery lifespan of the implantable infusion pump (e.g., 7-10 years) allows the device to remain implanted with the body for long periods of time without replacement. As compared with previous systems that are substantially housed external to the body of the patient, the ability to implant the present system within the body for multiple years allows the patient to not be hindered from performing common activities, such as swimming and taking baths.

Additionally, the present system allows for automatic, regular adjustment of insulin levels in response to periodic blood glucose measurements. Specifically, the glucose measurement device (e.g., impedance sensor) of the system can be configured to take periodic or real-time measurements without requiring prompting from the patient or medical professional attending to the patient. As such, in response to these periodic or real-time measurements, the present system can be automatically configured to release an effective amount of medication (e.g., insulin) into the portal vein in response to the determined glucose levels. Specifically, because the infusion pump is operatively coupled (wired, wirelessly e.g., Bluetooth) to the control unit, the control unit, upon receiving the glucose readings, can transmit signals to the pump including instructions for dose delivery of the medication. Accordingly, the present system, in operation, functions like an artificial pancreas, as it is a closed loop system that reacts to regulate the glucose levels of the patient regardless of whether the patient is directly interacting with the system (e.g., via an app). This is particularly important for patients who are sick or obtunded and thus unable to manage a conventional insulin pump on their own. For such patients, the system can be configured to automatically assess the blood sugar of a patient and subsequently deliver micro boluses of the medication (insulin) to the patient to manage their glucose levels. The present system also negates the need for the patient to actively determine their bolus dosing before bed, as the system can make this determination automatically based on its regular glucose readings throughout the day.

In one embodiment, the method may involve use of feedback to select a frequency of sampling the impedance to determine the blood glucose level over time and triggering the delivery of a dosage of medication selected in response to the detected impedance (or glucose level) crossing a threshold. By monitoring the time intervals and the blood glucose levels before and after the delivery of dosage, the system may adjust the frequency of delivering a unit dose and/or the size of the unit dose, and/or the frequency of sampling of impedance, so as to determine a dosing regimen configured for the patient. Such a dosing regimen can be different in response to different patient activity levels, e.g., during sleep, exercise and normal activities. Also, the dosing regimen may vary so as to reduce the frequency of dosage delivery of a relatively larger dosage or increase the frequency of dosage delivery with a relatively smaller dosage, as deemed appropriate for the patient.

Because the present system can determine a patient's blood glucose levels and deliver insulin in the portal vein, the insulin dosage required, as needed, is substantially reduced compared to a conventional subcutaneous injection, nasal inhalation, or external insulin pump. Specifically, in conventional methods, when insulin is injected subcutaneously or intravenously for example, much of it is absorbed or respired so that only a small proportion ends up passing into the portal vein and the liver where it is efficacious. In contrast, the present system delivers insulin directly to the portal vein, and thus a much smaller amount of insulin is required because there is minimal opportunity for the insulin to be absorbed before it reaches the liver, which is the organ in which insulin has its primary effects. The reduced insulin dosages can result in cost savings for the patient and less frequent refills of the reservoir, as well as smaller devices given the decreased volume requirement of the reservoir (as compared, for example, to the volume that would be needed for subcutaneous delivery over the same time period). Thus, patients may need less interaction with medical personnel, as they may be able to go a month or longer without needing a practitioner to refill the reservoir. Moreover, by virtue of being in the portal vein, the present system provides faster response times for controlling blood glucose levels within a narrower band of variation as compared with conventional treatments systems. Specifically, because glucose levels spike first in the portomesenteric circuit, the present system, by being in the portal vein, results in faster detection of those changes, and also a faster response given the proximity of the device to the liver site of insulin action.

Accordingly, because the present system is implantable and does not require the regular replacement or manipulation from the patient due to its long battery life, the present provides better overall control of glucose levels and a better patient experience.

Specific embodiments of the present application are further described in the items below.

Item 1. An implantable insulin pump system, comprising:
a subcutaneous implantable infusion pump comprising a reservoir of medication having an output tube, responsive to instructions to deliver a dose of a determined volume of the medication at a defined time;
a portal vein access catheter comprising:
   a catheter body having a proximal end, a distal end, a main lumen, wherein said main lumen extends from said proximal end to said distal end, and a balloon-fill lumen, wherein said balloon-fill lumen extends from a balloon-fill hub to an anchor balloon wherein said anchor balloon is insertable into a portal vein and inflatable to engage said portal vein and prevent dislodgement,
   a subcutaneous connection hub at said proximal end comprising an input port connected to said reservoir output tube and said balloon-fill hub, and
   a connector that interfaces said main lumen to said input port and said balloon-fill lumen to said balloon-fill hub;
a glucose measurement device configured to perform at least one measurement that is correlated with a patient's blood glucose levels;
a control unit operatively coupled to the infusion pump and the glucose measurement device, wherein the control unit is configured to determine the dosage of the medication to be delivered to the patient and determine a defined time of delivery of the dose via the pump into the portal vein of the patient.
   Item 2. The system of item 1, wherein the control unit is implantable.
   Item 3. The system of item 1, wherein the control unit is located outside of the body of the patient.
   Item 4. The system of items 1-3, wherein the medication is insulin or an insulin mimetic.
   Item 5. The system of items 1-4, wherein the reservoir is refillable.
   Item 6. The system of items 1-5, wherein at least one of the reservoir, infusion pump, and glucose measurement device is operatively coupled to the control unit via a wire connection.
   Item 7. The system of items 1-5, wherein at least one of the reservoir, infusion pump, and glucose measurement device is operatively coupled to the control unit via a wireless connection.
   Item 8. The system of items 1-7, wherein the glucose measurement device is associated with the catheter and comprises a transducer, and wherein glucose measurement device is configured to perform at least one measurement to determine a patient's blood glucose levels in the portal vein.
   Item 9. The system of item 8, wherein the transducer comprise two leads secured proximate to the tip of the catheter, wherein the leads are configured to measure impedance in the blood flow of the patient, which is correlated with the patient's blood glucose levels.
   Item 10. The system of item 9, wherein the leads of the glucose measurement device run the length of the catheter and attach to the control unit.
   Item 11. The system of items 8-10, wherein the glucose measurement device is a micro circuit and the transducer is integrated into the catheter.
   Item 12. The system of item 11, wherein the transducer is integrated into the micro circuit and the micro circuit is located at the tip of the catheter.
   Item 13. The system of items 1-12, wherein the glucose measurement device is an impedance sensor that is configured to measure an impedance in the blood flow which is correlated to a patient's blood glucose levels, and wherein the glucose measurement device or the control unit is configured to calculate a blood glucose level of the patient based on the impedance measurement.
   Item 14. The system of items 1-7, wherein the glucose measurement device is a fingerstick glucose device and an accompanying blood glucose meter.
   Item 15. The system of item 14, wherein the fingerstick glucose device comprises a lancet and a test strip, and wherein the blood glucose meter is operatively coupled to the control unit and configured to send glucose measurements to the control unit.
   Item 16. The system of items 1-7, wherein the glucose measurement device is a continuous glucose monitoring sensor.
   Item 17. The system of item 16, wherein at least a portion of the continuous glucose monitoring sensor is positioned subcutaneously and said portion is in fluid connection with the interstitial fluid of the patient.
   Item 18. The system of items 16-17, wherein the continuous glucose monitoring sensor is operatively coupled to the control unit and configured to send at least one measurement to the control unit that is correlated with the patient's blood glucose levels.
   Item 19. The system of items 1-11, further comprising an adaptor configured to connect the catheter to the infusion pump and keep the anchor balloon inflated.
   Item 20. The system of items 1-19, wherein the subcutaneous connection hub further comprises an infusion lumen port connected to said main lumen.
   Item 21. A method for regulating glucose in a subject with the implantable insulin pump system of items 1-20, the method comprising:
implanting at least a tip portion of a portal vein access catheter within a portal vein of a patient;
implanting an infusion pump subcutaneously, and coupling the infusion pump to the catheter;
conducting, with the glucose measurement device, a measurement on a fluid of the patient, wherein the fluid is interstitial fluid or blood, and wherein the measurement is correlated with a blood glucose level of the patient;
calculating, by the control unit, a dosage of insulin to deliver to the patient based on the measurement on the fluid of the patient;
delivering, by the infusion pump, the calculated dosage of insulin to the tip of the portal vein access catheter, wherein the tip of the portal vein catheter is in fluid connection with the portal vein; and
expelling, by the portal vein access catheter, the insulin from the tip of the catheter into the portal vein.
   Item 22. The method of item 21 wherein a portion of the delivered insulin mixes with a portion of the patient's blood in the tip of the catheter prior to expulsion into the portal vein.
   Item 23. The method of item 21, further comprising: implanting a portion of the glucose measurement device within the portal vein of the patient prior to conducting the measurement with the glucose measurement device on the fluid of the patient.

Although much of the foregoing description has been directed to systems and methods for an implantable insulin pump system and blood glucose regulation, the systems and methods disclosed herein can be similarly deployed and/or implemented in scenarios, situations, and settings far beyond the referenced scenarios. It should be further understood that any such implementation and/or deployment is within the scope of the composition and methods described herein.

It is to be further understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms ““including,” “comprising,” or “having,” “containing,” “involving,” and variations thereof herein, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Notably, the figures and examples above are not meant to limit the scope of the present disclosure to a single implementation, as other implementations are possible by way of interchange of some or all the described or illustrated elements. Moreover, where certain elements of the present disclosure can be partially or fully implemented using known components, only those portions of such known components that are necessary for an understanding of the present disclosure are described, and detailed descriptions of other portions of such known components are omitted so as not to obscure the disclosure. In the present specification, an implementation showing a singular component should not necessarily be limited to other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present disclosure encompasses present and future known equivalents to the known components referred to herein by way of illustration.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings are shown accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure and equivalent structures and functions or steps.

What is claimed is:

1. An implantable insulin pump system, comprising:
a subcutaneous implantable infusion pump comprising a reservoir of medication having an output tube, responsive to instructions to deliver a dose of a determined volume of the medication at a defined time;
a portal vein access catheter comprising:
a catheter body having a proximal end, a distal end, a main lumen, wherein said main lumen extends from said proximal end to said distal end, and a balloon-fill lumen, wherein said balloon-fill lumen extends from a balloon-fill hub to an anchor balloon wherein said anchor balloon is insertable into a portal vein and inflatable to engage said portal vein and prevent dislodgement,
a subcutaneous connection hub at said proximal end comprising an input port connected to said reservoir output tube and said balloon-fill hub, and
a connector that interfaces said main lumen to said input port and said balloon-fill lumen to said balloon-fill hub;
a glucose measurement device configured to perform at least one measurement that is correlated with a patient's blood glucose levels;
a control unit operatively coupled to the infusion pump and the glucose measurement device, wherein the control unit is configured to determine the dosage of the medication to be delivered to the patient and determine a defined time of delivery of the dose via the pump into the portal vein of the patient.

2. The system of claim 1, wherein the control unit is implantable.

3. The system of claim 1, wherein the control unit is located outside of the body of the patient.

4. The system of claim 1, wherein the medication is insulin or an insulin mimetic.

5. The system of claim 1, wherein the reservoir is refillable.

6. The system of claim 1, wherein at least one of the reservoir, infusion pump, and glucose measurement device is operatively coupled to the control unit via a wire connection.

7. The system of claim 1, wherein at least one of the reservoir, infusion pump, and glucose measurement device is operatively coupled to the control unit via a wireless connection.

8. The system of claim 1, wherein the glucose measurement device is associated with the catheter and comprises a transducer, and wherein glucose measurement device is configured to perform at least one measurement to determine a patient's blood glucose levels in the portal vein.

9. The system of claim 8, wherein the transducer comprises two leads secured proximate to the tip of the catheter, wherein the leads are configured to measure impedance in the blood flow of the patient, which is correlated with the patient's blood glucose levels.

10. The system of claim 9, wherein the leads of the glucose measurement device run the length of the catheter and attach to the control unit.

11. The system of claim 8, wherein the glucose measurement device is a micro circuit and the transducer is integrated into the catheter.

12. The system of claim 11, wherein the transducer is integrated into the micro circuit and the micro circuit is located at the tip of the catheter.

13. The system of claim 1, wherein the glucose measurement device is an impedance sensor that is configured to measure an impedance in the blood flow which is correlated to a patient's blood glucose levels, and wherein the glucose measurement device or the control unit is configured to calculate a blood glucose level of the patient based on the impedance measurement.

14. The system of claim 1, wherein the glucose measurement device is a fingerstick glucose device and an accompanying blood glucose meter.

15. The system of claim 14, wherein the fingerstick glucose device comprises a lancet and a test strip, and wherein the blood glucose meter is operatively coupled to the control unit and configured to send glucose measurements to the control unit.

16. The system of claim 1, wherein the glucose measurement device is a continuous glucose monitoring sensor.

17. The system of claim 16, wherein at least a portion of the continuous glucose monitoring sensor is positioned subcutaneously and said portion is in fluid connection with the interstitial fluid of the patient.

18. The system of claim 16, wherein the continuous glucose monitoring sensor is operatively coupled to the control unit and configured to send at least one measurement to the control unit that is correlated with the patient's blood glucose levels.

19. The system of claim 1, further comprising an adaptor configured to connect the catheter to the infusion pump and keep the anchor balloon inflated.

20. The system of claim 1, wherein the subcutaneous connection hub further comprises an infusion lumen port connected to said main lumen.

21. A method for regulating glucose in a subject with the implantable insulin pump system of claim 1, the method comprising:
implanting at least a tip portion of a portal vein access catheter within a portal vein of a patient;
implanting an infusion pump subcutaneously, and coupling the infusion pump to the catheter;
conducting, with the glucose measurement device, a measurement on a fluid of the patient, wherein the fluid is interstitial fluid or blood, and wherein the measurement is correlated with a blood glucose level of the patient;
calculating, by the control unit, a dosage of insulin to deliver to the patient based on the measurement on the fluid of the patient;
delivering, by the infusion pump, the calculated dosage of insulin to the tip of the portal vein access catheter, wherein the tip of the portal vein catheter is in fluid connection with the portal vein; and
expelling, by the portal vein access catheter, the insulin from the tip of the catheter into the portal vein.

22. The method of claim 21 wherein a portion of the delivered insulin mixes with a portion of the patient's blood in the tip of the catheter prior to being expelled into the portal vein.

23. The method of item 21, further comprising: implanting a portion of the glucose measurement device within the portal vein of the patient prior to conducting the measurement with the glucose measurement device on the fluid of the patient.
